SEQUENCE LISTING (2 sequences)

SEQ ID NO: 1 - DNA, 4898 bp, Artificial Sequence
  recombination plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgagctcggtacccgggcgaattccaagcttcaccagagaatacccagactgtgatgctgccacagtgtcagcaggctttctgaacggtataacaccacttcatgttaatgataattactatcattaaatcttgacatgccattttctccttaataaattagtactgtatatgtatccatatacgtaagcagttaattcatttgttttcctcagaggatgaaggagataccgaatgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgatcgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatcggatccagatctcatcaccatcaccatcactaagcttaattagctgagcttggactcctgttgatagatccagtaatgacctcagaacagatctggatcccctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 1890 bp, Escherichia coli
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgatcgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatcggatccagatct

(19) United States
(12) Patent Application Publication
(10) Pub. No.: US 2018/0042990 A1
(43) Pub. Date: Feb. 15, 2018

(54) METHOD FOR IMPROVING AN INTESTINAL MICROFLORA AND PROMOTING GROWTH OF ANIMALS BY USING COLICIN OR MICROORGANISM CAPABLE OF EXPRESSING THE SAME

(71) Applicant: National Chung Hsing University, Taichung (TW)

(72) Inventors: Jiann-Hwa CHEN, Taichung (TW); Chuan-Shun LIN, Miaoli County (TW)

(21) Appl. No.: 15/674,766

(22) Filed: Aug. 11, 2017

(30) Foreign Application Priority Data
Aug. 15, 2016 (TW) 105125966

(51) Int. Cl. A61K 38/16; A61K 35/74

(52) U.S. Cl. CPC A61K 38/164; A61K 35/74

(57) ABSTRACT

The present invention discloses use of a colicin Ib and a microorganism expressing the colicin Ib, to improve animal's intestinal microflora, and an effect of improving the growth efficiency and the feed-to-meat rate is obtained. Accordingly, the colicin Ib and the microorganism expressing colicin Ib can be used as a meat-growth agent or as effective gradient for a composition for promoting growth of animals or improving intestinal microflora.

### BACKGROUND OF THE INVENTION

### Field of the Invention

The invention relates to the use of colicin, particularly relates to a method for improving an intestinal microflora and promoting growth of animals by using a colicin Ib or a microorganism capable of expressing the colicin Ib the same.

### Description of the Related Art

Because weaning piglets face variable factors including separation from the sow, mixing with other pigs, and changes in feeding environment and food, the appetites of the piglets are negatively affected; causing that weights of the piglets cannot keep continuous increase. Therefore, during the feeding process of the piglets, it has been constantly seeking a method for maintaining the weight gain rate of the weaning piglets to improve the market economic benefits.

Feeds with high palatability are typically selected or added with drugs in the current feeding practice. However, the feeds with high palatability are still unable to ensure the active intake of all the piglets or the weight gaining effect of the feed on the piglets. In addition, it is indicated from researches that although the addition of drugs improves the daily weight gain, based on the consideration of the food safety, the use of the drugs during the feeding process may make the consumers have health concerns, which is not helpful to improve the market economic value or even results in anxiety of the consumers. Thus, most countries tend to limit the use of drugs in animal husbandry by relatively strict regulations, and it is desired to develop a method that takes into consideration of both food safety and animal growth.

### SUMMARY OF THE INVENTION

In view of the above-described problems, it is one objective of the invention to provide a method for using a colicin Ib to promote growth of animals or/and improve an intestinal microflora of animals. The colicin Ib can be used as a meat-growth agent and an intestinal microflora-improving agent to achieve the efficacy in improvement of the growth efficiency of animals.

It is another objective of the invention to provide a method for using a colicin Ib or a microorganism capable of expressing the colicin Ib, in which, effective proteins having small molecules substitute the pharmaceutical growth auxiliary agent, not only is the feed-to-meat rate improved, but also the meat quality is enabled to satisfy the standard of food safety, thereby improving the market economic value.

To achieve the above objects, in accordance with one embodiment of the invention, there is provided with a method for preparing a meat-growth agent by using a colicin Ib or a microorganism capable of expressing the colicin Ib, in which, a nucleotide sequence of the colicin Ib is a sequence represented by SEQ ID NO. 2 or a homologous nucleotide sequence derived from the sequence represented by SEQ ID NO. 2 by substitution, deletion, or addition of one or multiple nucleotides.

In one embodiment, the nucleotide sequence of colicin Ib has a similarity of greater than 90% to SEQ ID NO. 2. For example, the nucleotide sequence of the colicin Ib is SEQ ID NO. 2.

By administering an effective dose of a colicin Ib or a microorganism capable of expressing the colicin Ib to an animal, it can improve the intestinal microflora and increasing the feed conversion rate of the animal.

Furthermore, the intestinal microflora of the animal is improved by increasing an amount of at least a first intestinal bacterium and/or decreasing an amount of at least a second intestinal bacterium, wherein:

The first intestinal bacterium is Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia, or Mesoplasma entomophilum; and the second intestinal bacterium is Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii, or Pelagicoccaceae pelagicoccus.

In accordance with another embodiment of the invention, there is provided with a method for preparing a composition for promoting growth of animals by using a colicin Ib or a microorganism capable of expressing the colicin Ib, in which, a nucleotide sequence of the colicin Ib is a sequence represented by SEQ ID NO. 2 or a homologous nucleotide sequence derived from the sequence represented by SEQ ID NO. 2 by substitution, deletion, or addition of one or multiple nucleotides.

Furthermore, the nucleotide sequence of colicin Ib has a similarity of greater than 90% to SEQ ID NO. 2. For example, the nucleotide sequence of the colicin Ib is SEQ ID NO. 2.

In accordance with still another embodiment of the invention, there is provided with a method for preparing an intestinal microflora-improving agent for improving intestinal microflora by using a colicin Ib or a microorganism capable of expressing the colicin Ib, wherein a nucleotide sequence of the colicin Ib is a sequence represented by SEQ ID NO. 2 or a homologous nucleotide sequence derived from the sequence represented by SEQ ID NO. 2 by substitution, deletion, or addition of one or multiple nucleotides.

For example, the nucleotide sequence of colicin Ib has a similarity of greater than 90% to SEQ NO. 2.

In another embodiment, the nucleotide sequence of the colicin Ib is SEQ ID NO. 2.

The intestinal microflora-improving agent is capable of increasing an amount of at least one type of intestinal bacterium, the intestinal bacterium is Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia or Mesoplasma entomophilum.

Furthermore, the intestinal microflora-improving agent is capable of decreasing an amount of at least one type of intestinal bacterium, the intestinal bacterium is Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii or Pelagicoccaceae pelagicoccus.

Also, the above intestinal microflora-improving agent or meat-growth agent comprises at least one carrier, and a ratio of a dose of the carrier to a dose of the colicin Ib is 5×10⁴:2. For example, the carrier is starches, lipids, vitamins, a minerals, amino acids, proteins, or a combination thereof.

In accordance with still another embodiment of the invention, there is provided with a method for improving an intestinal microflora, comprising administering an effective dose of a colicin Ib or a microorganism capable of expressing the colicin Ib to an animal to increase an amount of at least a first intestinal bacterium and/or decrease an amount of at least a second intestinal bacterium, in which:

a nucleotide sequence of the colicin Ib is a sequence represented by SEQ ID NO. 2 or a homologous nucleotide sequence derived from the sequence represented by SEQ ID NO. 2 by substitution, deletion, or addition of one or multiple nucleotides;

the first intestinal bacterium is Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia or Mesoplasma entomophilum; and

the second intestinal bacterium is Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii or Pelagicoccaceae pelagicoccus.

The microorganism capable of expressing the colicin Ib is a recombinant microorganism constructed by genetic engineering and comprising the carrier having a sequence represented by SEQ ID NO. 2. Thus, the microorganism is able to produce the colicin Ib in a predetermined growth condition.

### BRIEF DESCRIPTION OF THE DRAWINGS

The invention is described hereinbelow with reference to the accompanying drawings, in which:

FIG. 1 is a sodium dodecyl sulfate-polyacrylamide gel electrophoresis (SDS-PAGE) chart;

FIG. 2 is a histogram indicating a daily weight gain of each group on different feeding days;

FIG. 3 is a histogram indicating a daily food intake of each group on different feeding days;

FIG. 4 is a histogram indicating a feed-to-meat rate of each group on different feeding days;

FIG. 5 is a histogram indicating a content of IL-2 in a blood of each group on different feeding days;

FIG. 6 is a histogram indicating a content of IgG in a blood of each group on different feeding days;

FIG. 7 is a histogram indicating a content of IgA in a blood of each group on different feeding days;

FIG. 8 is a histogram indicating a content of IFN-γ in the blood of each group on different feeding days;

FIG. 9A illustrates analyses of change of an intestinal microflora after different feeding conditions, in which, bacteria numbered between 1-28 having a difference value before and after feeding experiment of greater than 1 are indicated;

FIG. 9B illustrates the analyses of change of an intestinal microflora after different feeding conditions, in which, bacteria numbered between 29-53 having a difference value before and after feeding experiment of greater than 1 are indicated;

FIG. 10 illustrates the analyses of the change of an intestinal microflora after different feeding conditions, in which, 13 types of bacteria having a difference value before and after feeding experiment of greater than 4 are indicated;

FIG. 11 illustrates analyses results of FIGS. 9A-9B; and

FIG. 12 illustrates analyses results of FIG. 10.

### DETAILED DESCRIPTION OF THE EMBODIMENTS

For further illustrating the invention, experiments detailing a method for improving an intestinal microflora and promoting growth of animals by using a colicin Ib or a microorganism capable of expressing the colicin Ib the same. Furthermore, by administering an effective dose of a colicin Ib or a microorganism capable of expressing the colicin Ib to the animal, it can improve the intestinal microflora of the animal and increase the feed conversion rate of the animal. Therefore, it can make the animal grow faster without using antibiotic.

It should be noted that the following examples are intended to describe and not to limit the invention.

The colicin Ib or a homologous protein thereof disclosed by the invention can be acquired by techniques including extraction, artificial synthesis, and recombinant biological platform.

A plurality of examples are used to describe the effect of the invention hereinbelow.

### Example 1 Preparation of Protein Powder of Colicin Ib-His

In this example, the protein powder of the colicin Ib-His was prepared as follows:

1. A pT-ColIb-C-his-ok/BW251132 bacterium was constructed, in which, a sequence of a plasmid pT-ColIb-C-his-ok is represented by SEQ ID NO. 1, and a DNA sequence of the colicin Ib is represented by SEQ ID NO. 2.

2. Fresh colonies of the pT-ColIb-C-his-ok/BW251132 bacterium were inoculated to 5 mL of a first LB culture medium containing Ap50 and then cultured overnight.

3. One percent amount of the first LB culture medium comprising the bacteria was inoculated to 100 mL of a second LB culture medium containing Ap50 and then cultured overnight.

4. A solution comprising 0.5 mg/mL mitomycin C was added to the second LB culture medium to make a final concentration of the mitomycin C to be 0.2 μg/mL, and thereafter the bacteria was cultured in the second LB culture medium for 6 hrs.

5. The bacteria were centrifuged and collected, and stood at a temperature of −20° C. at least overnight.

6. The bacteria were collected and ultrasonically crushed. A resulting mixture was centrifuged and filtered so as to purify the protein colicin Ib-His.

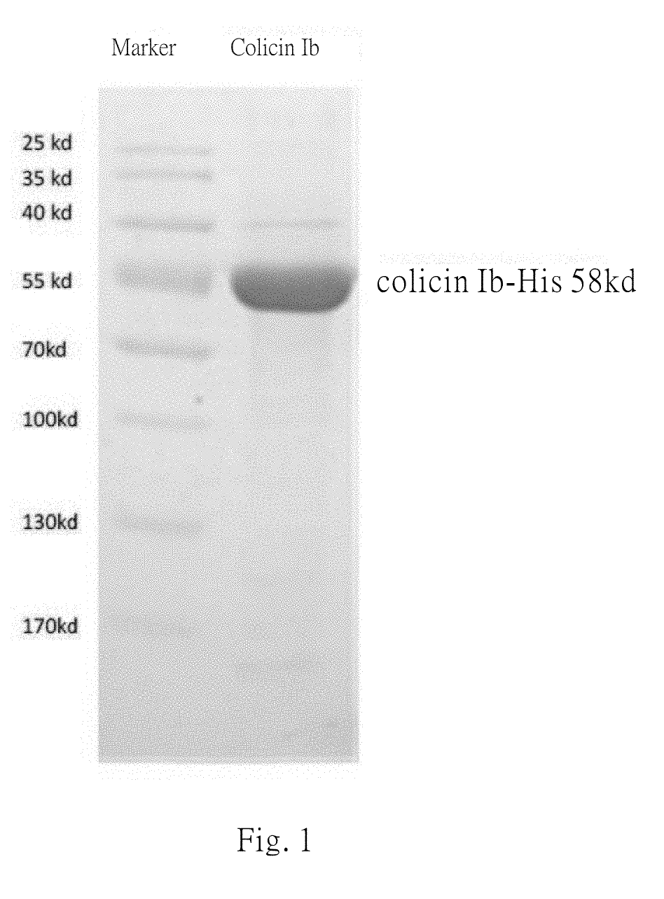

7. Acquired fractions were performed with 10% SDS-PAGE and western hybrid with anti-His antibody, results of which are illustrated in FIG. 1.

8. A fraction of the protein colicin Ib-His was collected and dialyzed using a 20 mM Tris buffer (pH 8.0).

9. A concentration of the protein colicin Ib-His and a volume of a dialysate were measured to calculate an amount of the protein colicin Ib-His.

10. The dialysate was lyophilized into powder which was then stored at a temperature of 4° C.

### Example 2 Animal Experiment

18 weaning piglets were selected and divided into three groups, with each groups housed in six sheds, and each shed was housed with one piglet. Feeding conditions for different groups were as follows:

A first group was a blank control group fed with a normal feed.

A second group was fed with the normal feed and an antibiotic Tiamulin.

A third group was fed with the normal feed and the colicin Ib prepared in Example 1, and a dose of the colicin was 20 mg per kilogram of the normal feed.

The normal feed used herein is generally known by persons skilled in the art therefore will not be explained in detail about the ingredient thereof.

The piglets of different groups were fed for 4 days according to the above conditions, weight, daily feed amount, daily weight gain, and feed-to-meat rate of the piglets of each group were detected and analyzed, results of which are listed in Table 1.

TABLE 1
growth indicators of piglets of each group

| | | | |
|---|---|---|---|
| Item | First group | Second group | Third group |

Weight (kg)

| | | | |
|---|---|---|---|
| Day 0 | 10.80 | 10.75 | 10.75 |
| Day 4 | 12.75 | 13.05 | 13.13 |

Daily weight gain (kg)

| | | | |
|---|---|---|---|
| Days 0-4 | 0.488 | 0.575 | 0.596 |

Daily food intake (kg)

| | | | |
|---|---|---|---|
| Days 0-4 | 0.754 | 0.792 | 0.767 |

Feed-to-meat rate (daily food intake/daily weight gain)

| | | | |
|---|---|---|---|
| Days 0-4 | 1.547 | 1.377 | 1.287 |

It is known from Table 1 that the colicin Ib does not affect appetites of the piglets, in addition to improve the food intake of the piglets, the intake of the food can be converted into meat, thus effectively improving the growth rate and the weight of the piglets.

### Example 3 Animal Experiment

18 weaning piglets were selected and divided into three groups, with each groups housed in six sheds, and each shed was housed with one piglet. Feeding conditions for different groups were as follows:

A first group was a blank control group fed with a normal feed.

A second group was fed with the normal feed and an antibiotic Tiamulin.

A third group was fed with the normal feed and the colicin Ib prepared in Example 1, and a dose of the colicin was 20 mg per kilogram of the normal feed.

The normal feed used herein is generally known by persons skilled in the art therefore will not be explained in detail about the ingredient thereof.

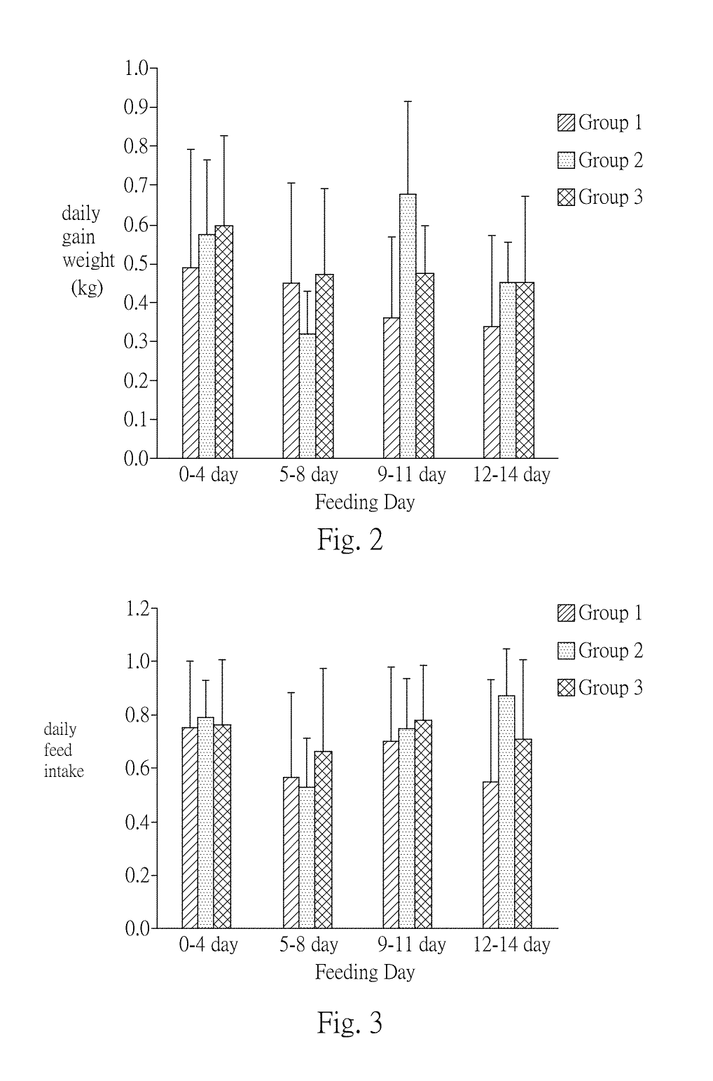

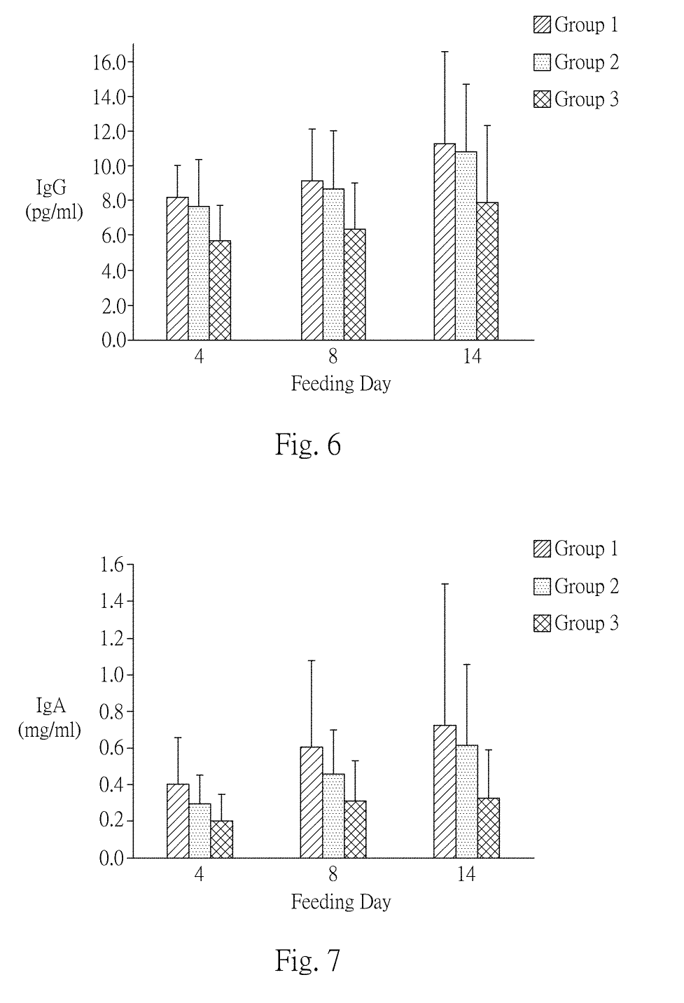

After the piglets of each group were fed according to the above conditions for 5 days, the piglets were administered with enterotoxigenic E. coli (ETEC), respectively, and then fed according to the above feeding conditions for another 10 days. During the whole process, the daily food intake of the piglets of each group was measured every day, and weights of the piglets and the content of the immune protein in the blood of the piglets of each group were measured at a fourth, eighth, eleventh, and fourteenth day, respectively, results of which are illustrated in FIGS. 2-8.

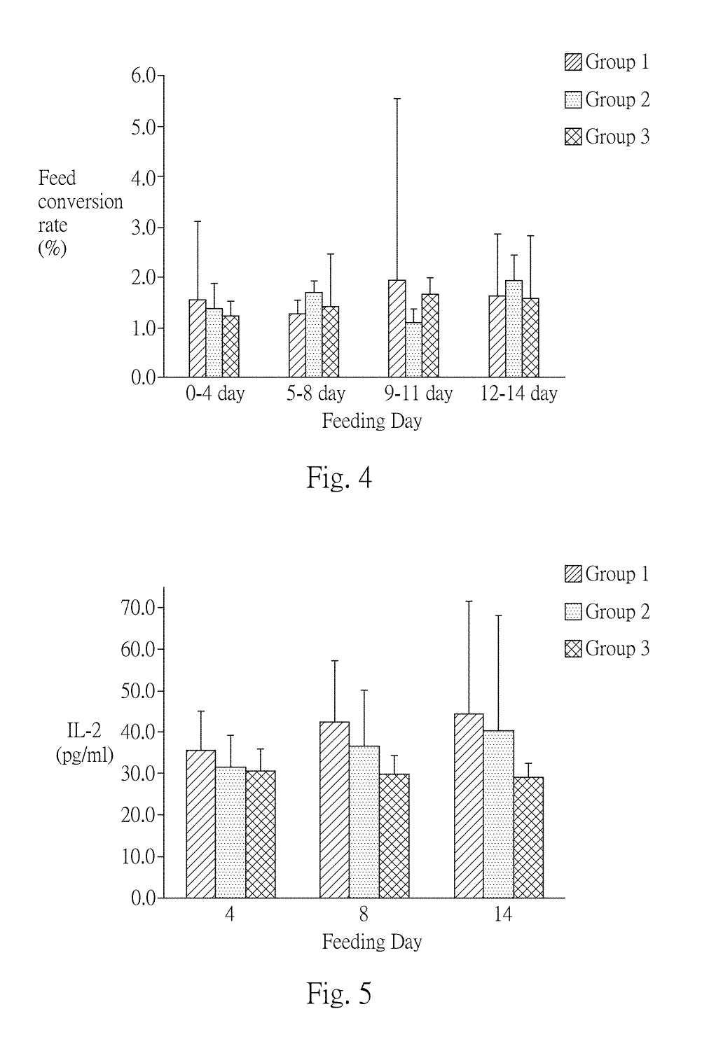

It is known from FIGS. 2-4 that after 14 days of feeding, the first group had a daily weight gain of approximately 0.427 kg and a feed utilization of approximately 1.649 wt. %; while the third group had a daily weight of 0.563 kg and a feed utilization of 1.473 kg. In another word, compared with the first group, the third group had the daily weight gain of the piglets improved by approximately 32 wt. % and the feed utilization decreased by 11%. Thus, after fed with the colicin disclosed by the invention, the daily weight gain of the piglets can be stably improved on the premise of not increasing the feed utilization and the food intake, thereby realizing the similar effect of feeding the antibiotics. However, it is known from the feed-to-meat rate (daily food intake/daily weight gain) of each group in FIG. 4 that long-term feeding of the colicin disclosed by the invention does not affect the appetite of the piglets but can improve the feed-to-meat rate of the piglets.

Furthermore, it was known from FIGS. 5-8 that the feeding of the colicin disclosed by the invention is able to reduce the immune proteins IgG, IgA, and IL-2 in the blood, and it was indicated that the colicin will not cause allergic reactions to the piglets.

### Example 4 Microflora Analyses

The weaning piglets were divided into two groups, and fecal materials of the piglets of each group were gathered before the feeding experiment. Thereafter, the piglets of the two groups were respectively fed by a normal feed in the absence of additive and by a normal feed added with the colicin disclosed by the invention for 4 days, the fecal materials of the piglets of each group were gathered again. A dose of the colicin was 20 mg per kilogram of the normal feed.

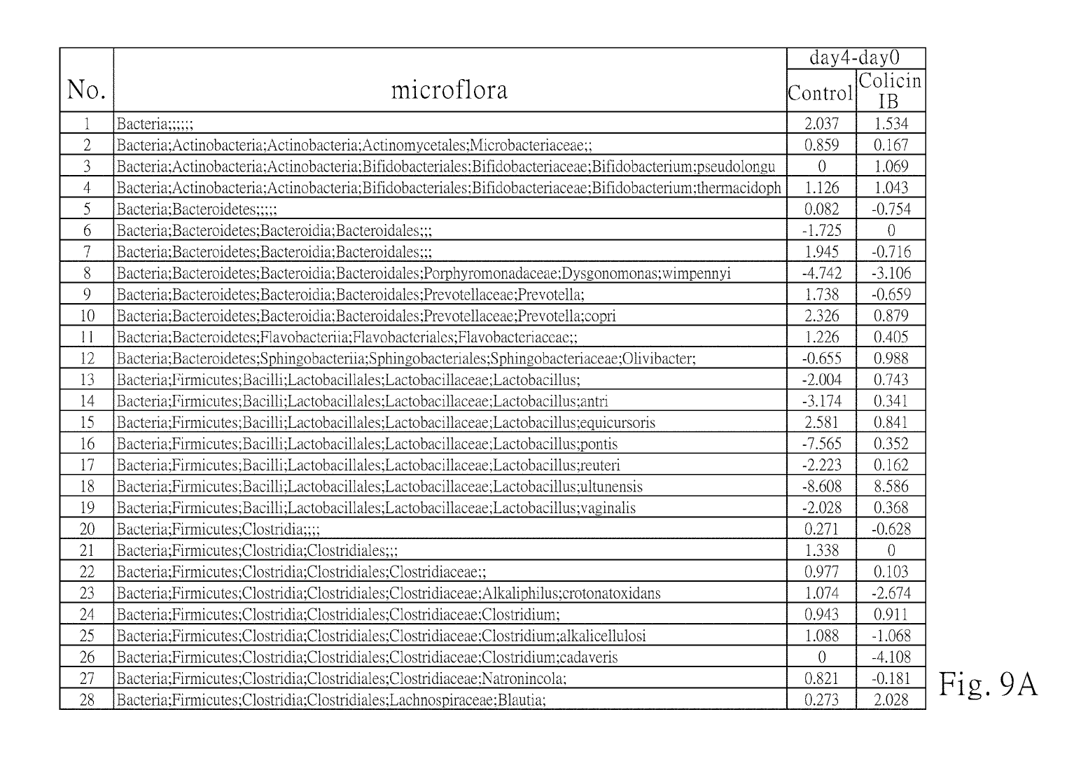

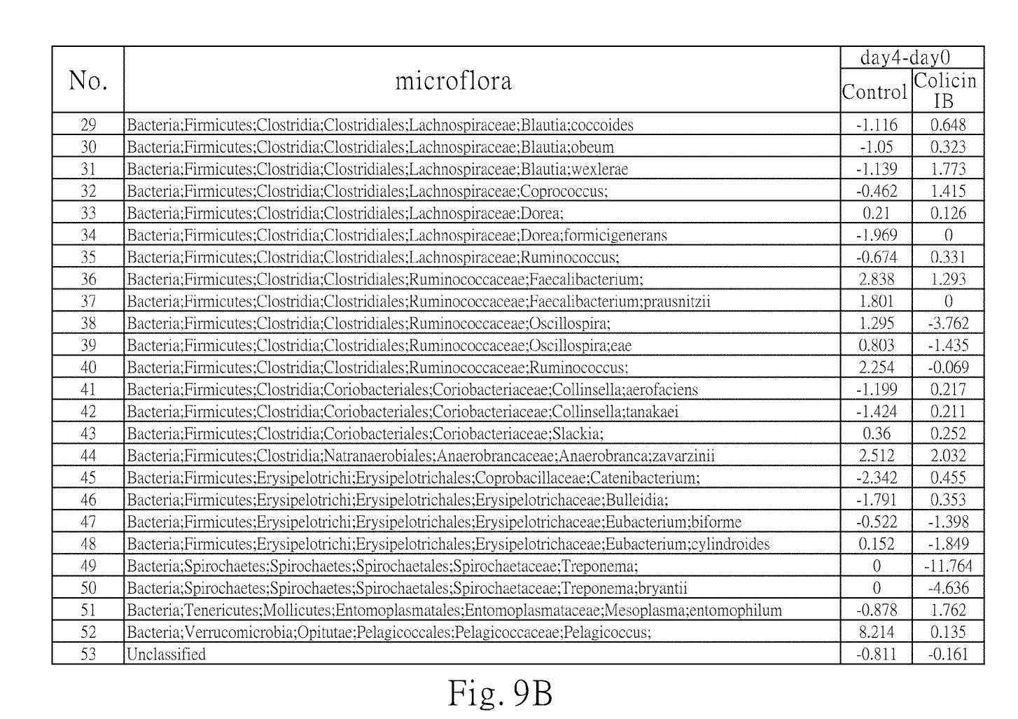

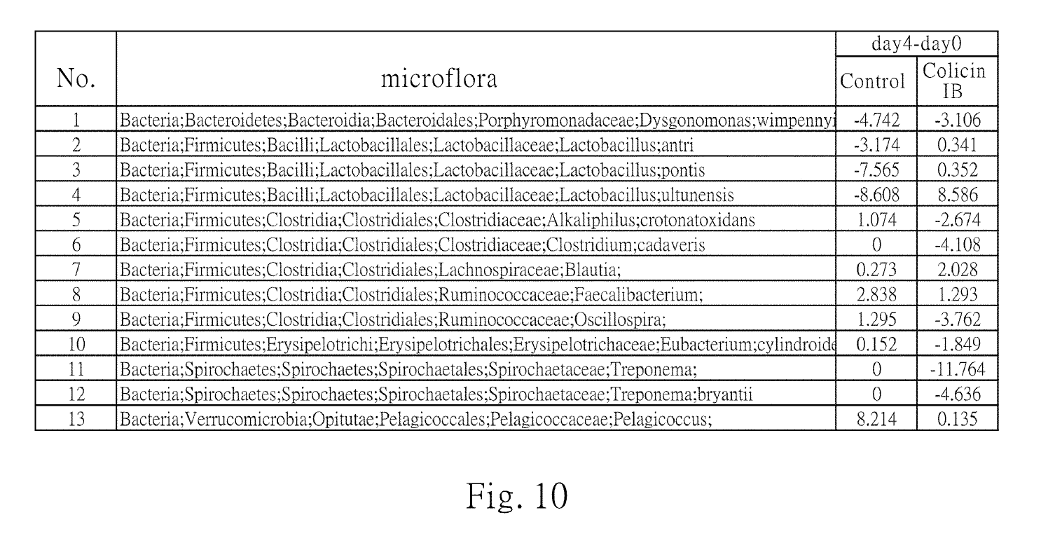

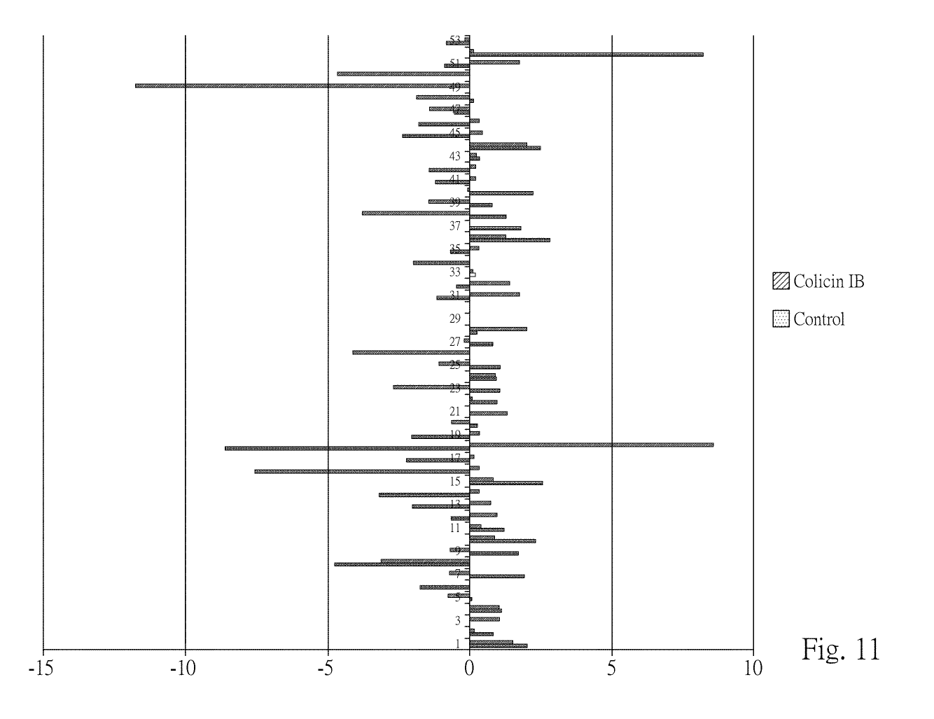

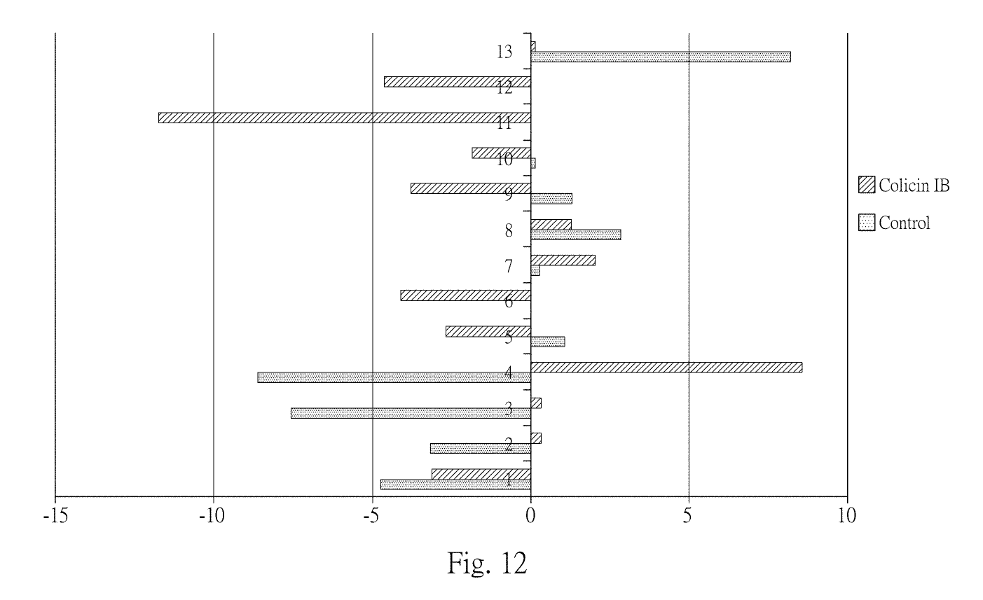

The fecal materials of the piglets of each group gathered respectively at the 0 day (before the feeding experiment) and at the fourth day were performed with microflora analyses. The ratios of different types of bacteria of a blank control group analyzed at the 0 day were respectively subtracted from the ratios of different types of bacteria of the blank control group analyzed at the fourth day; the ratios of different types of bacteria of the colicin group analyzed at the 0 day were respectively subtracted from the ratios of different types of bacteria of the colicin group analyzed at the fourth day; and only the bacteria with difference values of the ratios greater than 1 and 4 were reserved, results of which are listed in FIGS. 9-10. Results of FIGS. 9-10 were represented by histograms as shown in FIGS. 11-12.

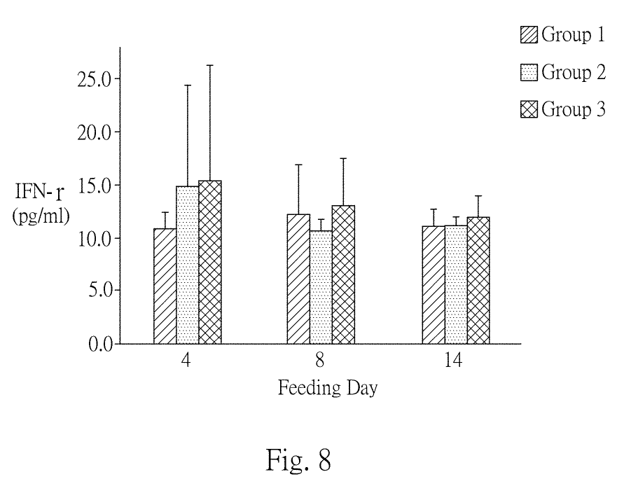

It is known from FIGS. 9-12 that the intestinal bacteria in the piglets fed with the colicin are different from the intestinal bacteria in those not fed with the colicin. Furthermore, it is clearly indicated from the results of FIGS. 8-11 that the feeding of the colicin is able to greatly increase the amount of intestinal bacteria comprising, for example, Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia, Mesoplasma entomophilum as well as reduce the amount of the intestinal bacteria comprising, for example, Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii, Pelagicoccaceae pelagicoccus, in which the increased amount of Lactobacillus ultunensis is the greatest.

Combining with the results of Examples 2-4, it can be inferred that the increase in the amount of the bacteria comprising Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia, Mesoplasma entomophilum and the decrease in the amount of the bacteria comprising Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii, Pelagicoccaceae pelagicoccus are related to the feed-to-meat rate of the piglets. In another word, the bacteria, such as Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia, Mesoplasma entomophilum are beneficial to improve the fee-to-meat rate, while the bacteria, such as Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii, Pelagicoccaceae pelagicoccus are harmful to increase the feed-to-meat rate.

In summary, when the colicin disclosed by the invention was fed to the piglets, by changing the intestinal microflora, the growth of the intestinal bacteria that are beneficial to the growth of the meat are facilitated, so that on the basis of maintaining the original food intake of the piglets, the food and energy intake can be effectively converted into meat, thereby realizing the efficacy in improvement of the growth efficiency of animals and decreasing or avoiding the weight reduction of the piglets due to changes of the growth environment. Therefore, the colicin or the microorganism capable of expressing the colicin Ib disclosed by the invention is able to substitute the antibiotics.

Unless otherwise indicated, the numerical ranges involved in the invention include the end values. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A method for promoting growth of animals, comprising administering an effective dose of a colicin Ib or a microorganism capable of expressing the colicin Ib to an animal to improve the intestinal microflora and increasing the feed conversion rate of the animal, wherein a nucleotide sequence of the colicin Ib is a sequence represented by SEQ ID NO. 2 or a homologous nucleotide sequence derived from the sequence represented by SEQ ID NO. 2 by substitution, deletion, or addition of one or multiple nucleotides.

2. The method of claim 1, wherein the nucleotide sequence of the colicin Ib is SEQ ID NO. 2.

3. The method of claim 1, wherein the nucleotide sequence of colicin Ib has a similarity of greater than 90% to SEQ ID NO. 2.

4. The method of claim 1, wherein the colicin Ib is mixed with at least one carrier and the ratio of a dose of the carrier to a dose of the colicin Ib is 2.5×10⁴:1.

5. The method of claim 1, wherein the intestinal microflora of the animal is improved by increasing an amount of at least a first intestinal bacterium and/or decreasing an amount of at least a second intestinal bacterium;
the first intestinal bacterium is selected from a group consisting of Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia, and Mesoplasma entomophilum; and
the second intestinal bacterium is selected from a group consisting of Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii, and Pelagicoccaceae pelagicoccus.

6. A method for improving an intestinal microflora of an animal, comprising administering an effective dose of a colicin Ib or a microorganism capable of expressing the colicin Ib to an animal to increase an amount of at least a first intestinal bacterium and/or decrease an amount of at least a second intestinal bacterium, wherein
a nucleotide sequence of the colicin Ib is a sequence represented by SEQ ID NO. 2 or a homologous nucleotide sequence derived from the sequence represented by SEQ ID NO. 2 by substitution, deletion, or addition of one or multiple nucleotides;
the first intestinal bacterium is selected from a group consisting of Lactobacillus ultunensis, Lachnospiraceae blautia, Blautia wexlerae, Lachnospiraceae coprococcus, Lachnospiraceae ruminococcus, Coprobacillaceae catenibacterium, Erysipelotrichaceae bulleidia, and Mesoplasma entomophilum; and
the second intestinal bacterium is selected from a group consisting of Alkaliphilus crotonatoxidans, Clostridium alkalicellulosi, Faecalibacterium prausnitzii, Clostridium cadaveris, Oscillospira eae, Eubacterium biforme, Ruminococcaceae oscillospira, Eubacterium cylindroides, Spirochaetaceae treponema, Treponema bryantii, and Pelagicoccaceae pelagicoccus.

7. The method of claim 6, wherein the nucleotide sequence of the colicin Ib is SEQ ID NO. 2.

8. The method of claim 6, wherein the nucleotide sequence of colicin Ib has a similarity of greater than 90% to SEQ ID NO. 2.
